(19) United States
(12) Patent Application Publication
(10) Pub. No.: US 2006/0247664 A1
(43) Pub. Date: Nov. 2, 2006

(54) Micromachined tissue anchors for securing implants without sutures

(75) Inventors: Ellis Meng, Pasadena, CA (US); Yu-Chong Tai, Pasadena, CA (US); Damien C. Rodger, Los Angeles, CA (US); Po-Jui Chen, Pasadena, CA (US); Mark Humayun, Glendale, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignees: California Institute of Technology, Pasadena, CA (US); University of Southern California, Los Angeles, CA (US)

(21) Appl. No.: 11/371,219

(22) Filed: Mar. 7, 2006

Related U.S. Application Data
(60) Provisional application No. 60/659,520, filed on Mar. 8, 2005.

(51) Int. Cl. A61F 2/02; A61B 3/16

(52) U.S. Cl. 606/151; 600/398

(57) ABSTRACT

Systems and methods for attaching an implant device to tissue by mechanically (and non-invasively) anchoring the device to the tissue. The systems and methods provide a safe, practical way to attach an implant device to tissue in a non-invasive, or less invasive manner. According to the present invention, an implant device includes one or more protruding anchor-like structures for securely attaching to tissue. One or more device features, such as sensing elements, may be incorporated on the implant device. The anchor structures are configured and arranged to match the topology and features of the tissue environment where implant is to occur.

### CROSS-REFERENCES TO RELATED APPLICATIONS

This application claims the benefit of U.S. Provisional Application No. 60/659,520 (Attorney docket No. 020859-008300US; Client Ref. CIT-4325-P), filed Mar. 8, 2005, the disclosure of which is incorporated herein by reference in its entirety.

### STATEMENT AS TO RIGHTS TO INVENTIONS MADE UNDER FEDERALLY SPONSORED RESEARCH OR DEVELOPMENT

The government may have certain rights to the invention based on National Science Foundation Grant EEC-0310723.

### BACKGROUND OF THE INVENTION

The present invention relates generally to systems and methods for securing implants to tissue, and more particularly to implant structures and devices with tissue anchors for use in securing the implant structures to tissue without sutures.

Biomedical implants often require a means of attaching to the body in order to secure the device in the desired location. One such device might be an implantable passive sensor for determining intraocular pressure in the eye. In order to diagnose a patient's condition, an ophthalmologist may require visual inspection of the sensor's pressure indicator readout. Thus, the logical choice for placement of this sensor would be behind the transparent cornea on the iris to allow for visual inspection when needed. However, conventional methods of suturing the device to the iris are invasive and potentially damaging. Similarly, for implant devices designed for other tissue locations, suturing and other securing techniques are invasive and potentially damaging to surrounding tissue.

Therefore it is desirable to provide systems and methods for attaching an implant device to tissue that overcome the above and other problems. Such systems and methods should be safe, practical and non-invasive or less invasive than current procedures.

### BRIEF SUMMARY OF THE INVENTION

The present invention provides systems and methods for attaching an implant device to tissue by mechanically (and non-invasively) anchoring the device to the tissue. The systems and methods provide a safe, practical way to attach an implant device to tissue in a non-invasive, or less invasive manner.

According to the present invention, an implant device includes one or more protruding anchor-like structures for securely attaching to tissue. One or more device features, such as sensing elements, may be incorporated on the implant device. The anchor structures are configured and arranged to match the topology and features of the tissue environment where implant is to occur. In the case of an intraocular implant device, for example, the implant device is anchored to the surface of the iris. The surface topology of the iris includes numerous folds resembling hills and valleys. These complex features can capture and hold a structure, such as a flat plate with protruding, anchor-like structures on one-side, in place.

According to one aspect of the present invention, an implant device is provided that typically includes an implant structure, and one or more anchor structures protruding from a surface of the implant structure. In certain aspects, the implant structure includes an implant feature, such as a sensor element, formed or attached to the implant structure. In certain aspects, the anchor structures are integral with the surface and/or are attached to or bonded with the surface.

According to another aspect of the present invention, a method is provided for fabricating an implant device. The method typically includes providing a substrate, and forming one or more anchor structures on a first surface of the implant structure. In certain aspects, forming includes separately fabricating the one or more anchor structures, and attaching or bonding the one or more anchor structures to the first surface. In certain aspects, forming includes forming an oxide layer on the first surface, patterning the oxide layer, and etching the first surface to form the one or more anchor structures. In certain aspects, the method also includes forming a device feature, such as a sensor element, on the first or a second surface of the substrate.

According to yet another aspect of the present invention, a method is provided for securing an implant device to tissue. The method typically includes providing an implant structure having a plurality of anchor structures protruding from a surface of the implant structure, the anchor structures being adapted to conform to the tissue topology at an implant location, and securing the device to a tissue location by contacting the anchor structures to the tissue in the implant location. In certain aspects, the implant location includes one of an iris, a retina, or a sclera of an eye.

Reference to the remaining portions of the specification, including the drawings and claims, will realize other features and advantages of the present invention. Further features and advantages of the present invention, as well as the structure and operation of various embodiments of the present invention, are described in detail below with respect to the accompanying drawings. In the drawings, like reference numbers indicate identical or functionally similar elements.

### BRIEF DESCRIPTION OF THE DRAWINGS

FIG. 1 illustrates top and bottom perspective views of an example of an ocular pressure sensor implant device including tissue anchors according to one embodiment. Top and bottom views of the device are shown with tissue anchors protruding from the bottom of a flat substrate.

FIG. 2 illustrates a layout of 24 different anchor platforms according to one embodiment. As shown, there are 3 rows, each corresponding to a different shape anchor, and 8 columns each having a different layout, size, and density of anchors.

FIG. 3 illustrates a tilted view of fabricated arrays of square-shaped anchors according to one embodiment.

FIG. 4 illustrates a layout of 3 different anchor platforms according to one embodiment. Each layout has 3 anchors of the same size (e.g., circular: 250 μm diameter, square: 250 μm each side, radial arms: 8 arms each μm wide).

FIG. 5 illustrates fabricated (a) circular, (b) square, and (c) radiating arm anchor platforms, and close-up images of anchors according to one embodiment.

FIG. 6 illustrates a side view of an anchor platform showing anchors protruding from the backside of the platform according to one embodiment.

FIG. 7 shows the chemical structure of the three most common types of parylene.

FIG. 8 illustrates a process of fabricating an implant device with integral anchor structures according to one embodiment.

FIG. 9 is a micrograph cross-section side view of a fabricated anchor with a tapered profile.

FIG. 10 illustrates another process of fabricating an implant device with integral anchor structures according to one embodiment.

FIG. 11 illustrates another process of fabricating an implant device with integral anchor structures using a “soft-stamp” technique according to one embodiment.

FIG. 12 is a micrograph bottom view of fabricated anchors coated with parylene.

FIG. 13 is a picture illustrating a device anchored on human skin.

### DETAILED DESCRIPTION OF THE INVENTION

General

The present invention provides implant assemblies and devices including one or more tissue anchoring elements and methods for fabricating the same. The present invention also provides systems and methods for anchoring implant devices to tissue.

FIG. 1 illustrates top and bottom perspective views of an implant device 10 including a sensor 25 and tissue anchoring elements 20 according to one embodiment. Top and bottom views of the device are shown with tissue anchoring elements 20 protruding from the bottom of a flat platform 30, such as a silicon substrate or other substrate. A sensor 25, such as an intraocular pressure sensor, is located on the top portion of platform 30 as shown. The sensor 25 may be formed on platform 30 or attached to platform 30.

It should be understood that the anchoring assemblies, devices, systems and methods are not limited to ocular implant, but rather are useful for securing any diagnostic or therapeutic devices to tissue in various parts of the body by matching the geometry and dimensions of the anchoring elements (anchors) 20 according to the tissue surface topologies present at the desired implant location(s). In certain aspects, this may include making the supporting substrate and/or anchors conform to the three-dimensional surfaces to which they will attach. The anchoring system includes a supporting platform on which a device can be integrated and from which the anchoring members protrude. These platform structures, which may be flexible or inflexible, may have anchors on more than one surface to allow sufficient attachment force. In addition, the platform may contain features such as diagnostic and therapeutic devices, cosmetic features, identification features, and anchors. In general, the present invention allows for any small, light-weight structure to be attached to or implanted in the body without the use of sutures or other invasive or harmful securing techniques, such as tacking or stapling.

Design and Materials

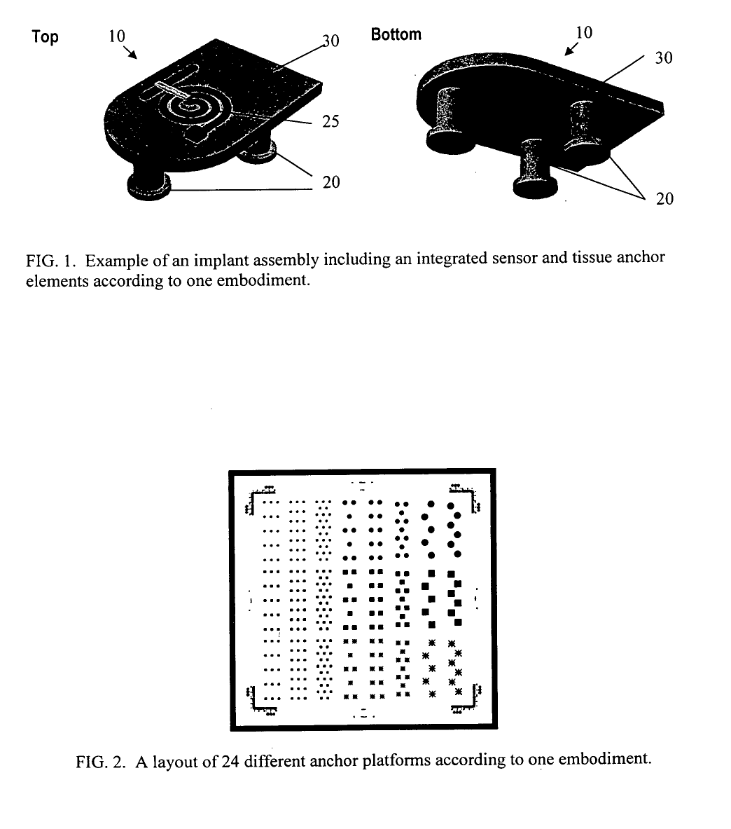

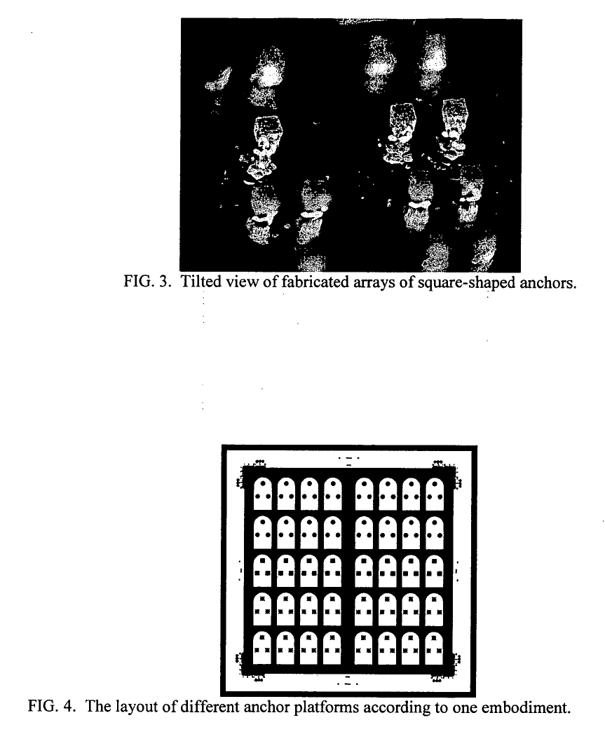

Several examples of designs of platforms and tissue anchor elements 20 for securing a device to tissue are presented in FIGS. 2-6. A specific layout of protruding anchors, anchor sizes, and anchor geometries will be discussed with reference to an iris implantation application, but these features can be used in, or can be adjusted for adaptation to, other applications. In one aspect, devices and components are coated with a biocompatible material such as parylene (poly-para-xylene), however other thin film biocompatible coatings can also be used. In one design, a long strip of silicon (e.g., 1 mm×2.5 mm) includes the pillar-like anchors (e.g., ˜0.25 mm length). The size, shape, layout, and density of anchors may be varied as shown in FIG. 2, which illustrates a layout of 24 different anchor platforms according to one embodiment. As shown, there are 3 rows, each corresponding to a different shape anchor, and 8 columns each having a different layout, size, and density of anchors. Examples of fabricated square anchors are shown in FIG. 3.

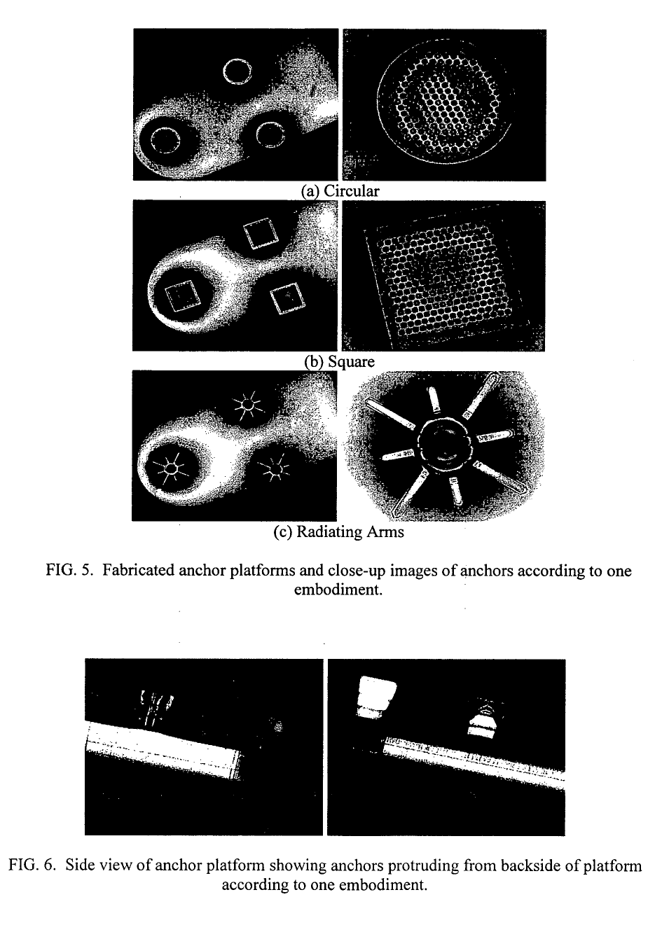

FIG. 4 illustrates a second design set, similar to FIG. 2, however the overall platform size (e.g., 0.75 mm×2 mm) is reduced to facilitate implantation. Also, the shape of the platform is rounded for easy insertion through an incision. As shown, each platform includes three anchors (e.g., ˜0.25 mm length) which is determined to be sufficient based on trial implantations. In certain aspects, 1, 2 or more anchors may be used. Examples of fabricated anchor platforms are shown in FIG. 5-6.

It should be understood that portions or all of a platform structure may be square or rectangular, polygonal, circular, elliptical etc. and that the platform structure may be inflexible or flexible. Also, the cross section of a peg or pillar defining an anchor 20 may be elliptical, circular and/or polygonal or any combination thereof throughout the length of the pillar. The number of sides of a polygonal cross-section may vary from 3 to about 16. One example is a four-sided polygon such as a square or rectangle. The sizes and dimensions of devices and features (e.g., platform, anchors, sensor, etc.) may vary. Possible and practical size ranges and dimensions of the platform, anchors and device features such as a sensor will generally depend on the body part and tissue to which the device will be adhered. For example, for the platform, dimensions in the mm-cm range are useful; for the anchors, dimensions in the μm-mm range and even into the cm range are useful It should be appreciated that other smaller or larger device dimensions may be used. Additionally, device features can include any of a variety of structures. One example of a useful feature is a sensor element including for example one or more of a pressure sensor, a temperature sensor, a shear stress sensor, a strain gauge, an optical sensor, a chemical sensor, a physical sensor, and a biosensor.

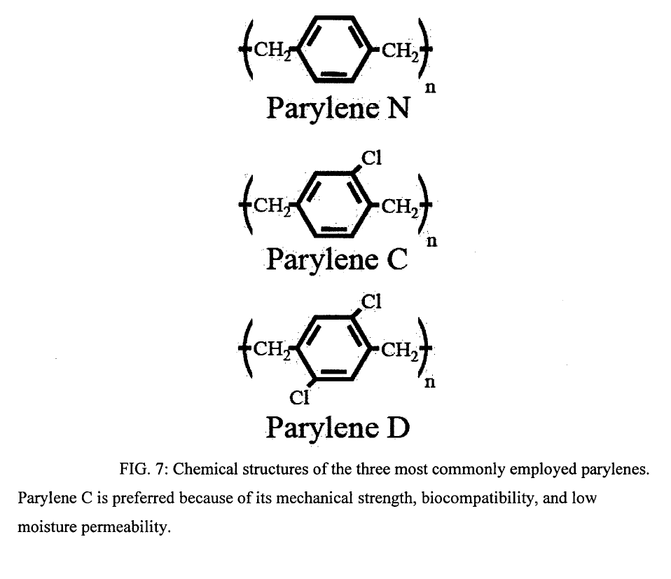

In certain aspects, to render devices and anchor structures biocompatible, it may be necessary to apply, or otherwise coat, the structures with a biocompatible material. One such biocompatible material is parylene (poly-para-xylene), which is a USP Class VI biocompatible material that has been approved for use in chronic implants, and has also been shown to be compatible with the intraocular environment. The conformality of the parylene deposition process also makes it ideal for use in hermetic sealing applications when device electronics must be shielded from the saline environment of the body. Parylene is also a very flexible, lightweight polymer and as such is optimal for matching anatomical morphology as well as for surgical implantation. Parylene can be deposited through a highly-conformal vapor deposition process. Types of commercially-available parylene include parylene C, F, A, AM, N, and D. Of the three most common types of parylene, shown in FIG. 7, parylene C is perhaps the most widely used in industry. The advantages of the use of parylene include its proven biocompatibility, its strength and flexibility (e.g., Young's modulus ≈4 GPa), its conformal pinhole-free room-temperature deposition, its low dielectric constant (≈3) and high volume resistivity (>10¹⁶ Ω-cm), its transparency, and its ease of manipulation using standard microfabrication techniques such as reactive ion etching (RIE).

Additional or alternative biocompatible materials might include biocompatible metals, such as gold (Au), titanium (Ti), platinum (Pt) and others; organic materials; biologically derived materials and adhesives; and inorganic materials and adhesives.

Fabrication

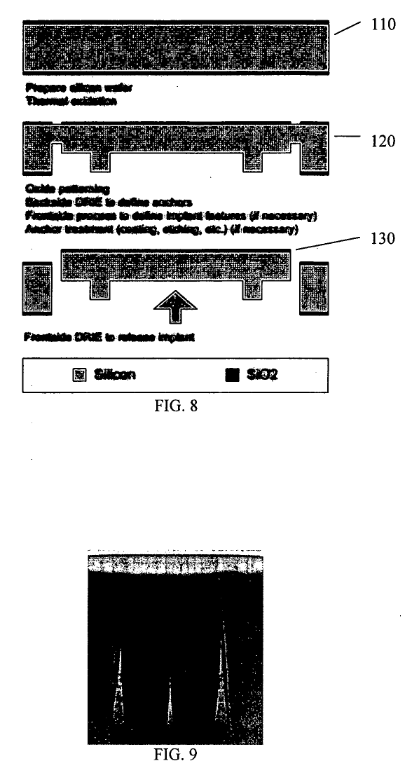

According to one embodiment, anchor elements are fabricated for the purposes of anchoring to tissue. In certain aspects, for example, anchor structures such as pegs or pillars, or pegs with the chair-like feet, can be microfabricated in either an integrated process or a micro-assembly process. Various examples of device fabrication methodologies are shown in FIGS. 8, 10 and 11. The fabrication processes described herein are but examples of many possibilities to machine anchors from materials such as silicon and parylene.

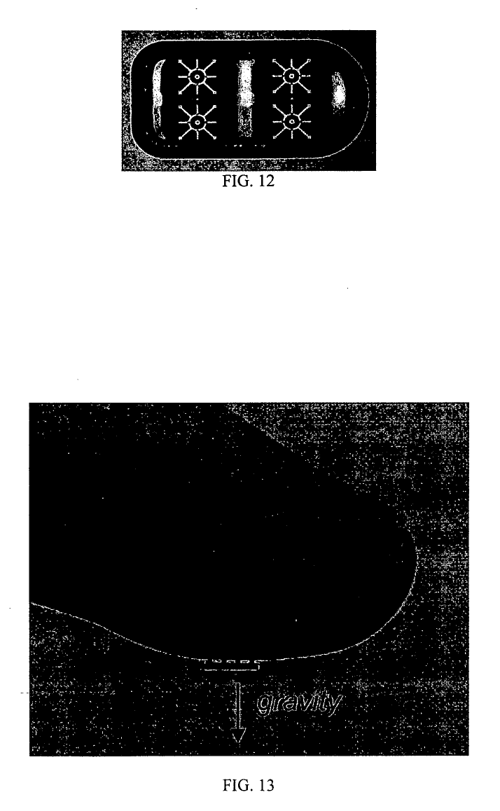

FIG. 8 illustrates an integrated micro-fabrication process for fabricating an implant device with integral anchor structures according to one embodiment. First, in step 110, a thermal oxide layer is formed or grown on a substrate. For example, a SiO₂ layer (e.g., >0.5 μm) may be formed by thermal oxidation of a silicon substrate/wafer. In step 120, an anchor pattern is transferred to the wafer using standard photolithographic techniques. The pattern may include a plurality of the same or different geometrically shaped anchor outlines. The anchor outlines on the backside are etched into the oxide layer, e.g., using a buffered oxide etch and a deep reactive ion etch (DRIE), to define the post structures that will serve as anchor elements. The frontside of the substrate (side opposite the anchor structures) may also be processed, e.g., to define implant features such as sensor features, if desired. If desired, the anchor/post structures can be undercut using wet or dry isotropic etching techniques such that the post is terminated by a slightly overhanging oxide etch mask. In step 130, the implant device is released, e.g., using a frontside DRIE. The anchoring posts and/or other device features can be optionally coated in a layer of biocompatible material such as parylene or other biocompatible materials to render them biocompatible either before or after step 130. FIG. 12 is a micrograph bottom view of fabricated chair like anchors coated with parylene.

In one aspect, during the backside DRIE, by controlling the parameters of the DRIE and implementing extensive SF₆ plasma etching, the anchor pegs can be etched to have a tapered profile. Depending on the tissue topology, a tapered profile may enhance the grabbing force of anchors to the attaching surface. FIG. 9 shows a micrograph cross-section side view of a fabricated anchor with a tapered profile. Additional treatments can be also done to the anchors to promote their physical and/or chemical adhesion with tissues. Examples of additional treatments might include coating an anchor element with an organic or inorganic adhesive. Other useful treatments include nano-particle or SAM (self-assembled monolayer) deposition.

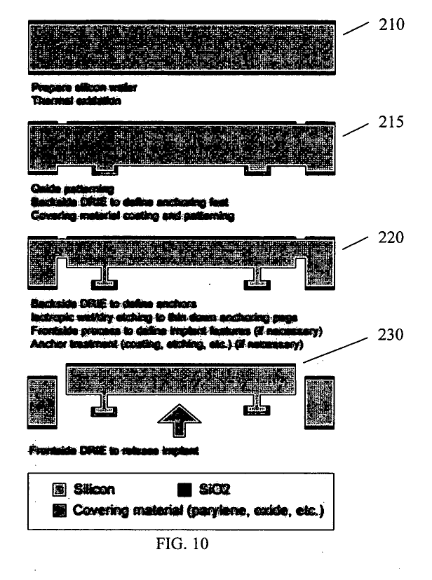

FIG. 10 illustrates another integrated microfabrication process for fabricating an implant device with integral chair-like anchor structures (e.g., structures having arms or feet radiating from the anchor post) according to one embodiment. In step 210, a thermal oxide layer is formed or grown on a substrate. For example, a SiO₂ layer (e.g., >0.5 μm) may be formed by thermal oxidation of a silicon substrate/wafer. In step 215, an anchor pattern is transferred to the wafer using standard photolithographic techniques. The pattern may include a plurality of the same or different geometrically shaped anchor outlines. The anchor outlines on the backside are etched into the oxide layer, e.g., using a buffered oxide etch or a deep reactive ion etch (DRIE) to define the arms or feet. A covering material is then applied to the arms or feet, which material also serves as an etch mask to preserve the arms or feet. The covering material may include parylene, oxide, photoresist, any high selectivity masking material, etc. In step 220, the anchor posts are defined by further DRIE or other etch. The posts may also be thinned down by extensive isotropic wet/dry silicon etching. However, the anchoring feet remain intact due to the protection of etch mask. In this way chair-like (straight pegs with radiating arms or feet) anchor structures can be fabricated. The frontside of the substrate (side opposite the anchor structures) may also be processed, e.g., to define implant features such as sensor features, if desired during steps 215 and/or 220. In step 230, the implant device is released, e.g., using a frontside DRIE. The anchoring posts and/or other device features can be optionally coated in a layer of biocompatible material such as parylene or other biocompatible materials to render them biocompatible. For example, if an oxide layer was used as an etch mask, a layer of parylene or other biocompatible material may be applied to the anchors and/or the entire device. If a parylene layer was used as an etch mask, a layer of parylene or other biocompatible material may be applied to the anchors and/or remaining features of the device.

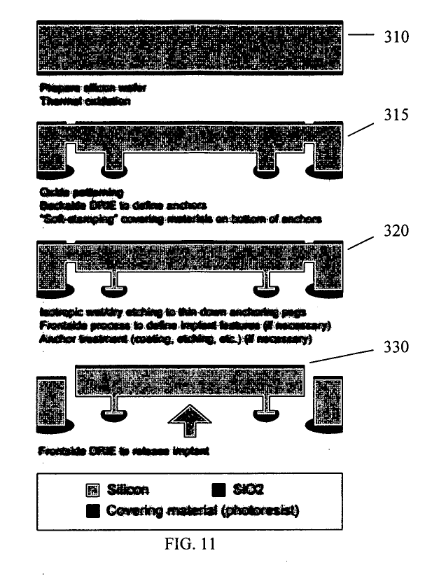

FIG. 11 illustrates another integrated microfabrication process for fabricating an implant device with integral anchor structures using a “soft-stamp” technique according to one embodiment. Similar to the process of FIG. 10, a “soft-stamp” technique is used to attach covering materials (e.g. photoresist or other viscous polymers before curing) on the bottom of the anchor structure(s), which may be thinned down by isotropic wet/dry etching. At the same time the bottom of the anchor structure(s) is still secured so radiating feet or other structures can be obtained. Process steps 310, 315, 320 and 330 are similar to steps 210, 215, 220 and 230 of FIG. 10. However, in step 315, anchoring feet features are covered with photoresist, and those covering materials are removable after the fabrication process. For biocompatibility, a biocompatible material such as parylene may be applied to the anchors and/or device after the anchor structures have been formed.

In certain aspects, anchoring pegs and feet can be separately fabricated on different substrates, then attached to an implant platform (e.g. by using thermal or anodic bonding or an adhesive) to construct implant assemblies with anchors. It is possible to use other materials in similar configurations to achieve the same result. For example, anchor structures such as pegs may be fabricated in whole or in part from glass or quartz, polymers or photo-definable polymers.

### EXAMPLES

Two generations of prototype intraocular implant devices similar to those described herein were implanted into rabbits and are being evaluated for adaptation to humans. In both versions of the anchors, the act of resting the anchors on top of a rabbit's iris was enough to hold the device in place. Although the precise removal force was not quantitatively determined, the mechanical locking of the anchors with the iris was more than sufficient to keep the devices secured to the iris. A significant amount of force is necessary to remove the devices once in place (such forces are greater than that exerted on the device during normal eye movement). FIG. 13 is a picture illustrating a device with anchors anchored on human skin. The device remains secured to the finger tissue even during serious shaking of the finger.

While the invention has been described by way of example and in terms of the specific embodiments, it is to be understood that the invention is not limited to the disclosed embodiments. To the contrary, it is intended to cover various modifications and similar arrangements as would be apparent to those skilled in the art. For example, anchors could be fabricated on curved or flexible substrates. Therefore, the scope of the appended claims should be accorded the broadest interpretation so as to encompass all such modifications and similar arrangements.

1. An implant device, comprising:
an implant structure; and
one or more anchor structures protruding from a surface of the implant structure.

2. The device of claim 1, further including an implant feature formed on or attached to the implant structure.

3. The device of claim 2, wherein the implant feature includes a sensor.

4. The device of claim 1, wherein the anchor structures are integral with the surface.

5. The device of claim 1, wherein the anchor structures are attached to or bonded to the surface.

6. The device of claim 1, further including at least one anchor structure protruding from a second surface of the implant structure.

7. The device of claim 6, wherein the at least one anchor structure is integral with the second surface.

8. The device of claim 6, wherein the at least one anchor structure is attached to or bonded to the second surface.

9. The device of claim 1, wherein the implant structure is flexible.

10. The device of claim 1, wherein the one or more anchor structures each comprise a pillar structure having one of a circular, elliptical or polygonal cross-section.

11. The device of claim 10, wherein at least a first one of the anchor structures has one or more radiating arms at an end distal from the implant structure.

12. A method of fabricating an implant device, comprising:
providing a substrate; and
forming one or more anchor structures on a surface of the implant structure.

13. The method of claim 12, wherein forming includes:
separately fabricating the one or more anchor structures; and
attaching or bonding the one or more anchor structures to the surface.

14. The method of claim 12, wherein forming includes:
forming an oxide layer on the surface;
patterning the oxide layer; and
etching the surface to form the one or more anchor structures.

15. The method of claim 14, further including:
forming a device feature on a second surface of the substrate.

16. The method of claim 15, wherein forming a device feature includes attaching or bonding the device feature to the second surface.

17. The method of claim 15, wherein forming a device feature includes processing the second surface to form all or a portion of the device feature.

18. The method of claim 15, wherein the device feature includes a sensor element selected from a group consisting of a pressure sensor, a temperature sensor, a shear stress sensor, a strain gauge, an optical sensor, a chemical sensor, a physical sensor, and a biosensor.

19-23. (canceled)

24. A method of securing an implant device to tissue, comprising:
providing an implant structure having a plurality of anchor structures protruding from a surface of the implant structure, said anchor structures being adapted to conform to the tissue topology at an implant location; and
securing the device to a tissue location by contacting the anchor structures to the tissue in the implant location.

25-29. (canceled)

30. The method of claim 24, wherein the implant location includes one of an iris, a retina or a sclera of an eye.
